(19) United States
(12) Patent Application Publication
(10) Pub. No.: US 2005/0124879 A1
(43) Pub. Date: Jun. 9, 2005

(54) Tissue imaging at superharmonic frequencies

(75) Inventors: Ayache Bouakaz, Rotterdam (NL); Nico De Jong, Krimpen aan den Ijssel (NL)

Correspondence Address: Kenton R Mullins;Stout Uxa Buyan & Mullins, Suite 300, 4 Venture, Irvine, CA, 92618, US

(73) Assignee: Stichting voor de Technische Wetenschappen, Utrecht (NL)

(21) Appl. No.: 10/502,077

(22) PCT Filed: Jan. 17, 2003
(86) PCT No.: PCT/EP03/00517

(30) Foreign Application Priority Data
Jan. 19, 2002 (GB) 0201239.1

(51) Int. Cl. A61B 8/00

(52) U.S. Cl. 600/437

(57) ABSTRACT

Ultrasound imaging of tissue in medical applications is carried out by transmitting ultrasound energy into a target volume at least a first fundamental frequency, receiving reflected and/or scattered ultrasound energy from the target volume; and detecting components of the received signal at multiple harmonics of the fundamental frequency or at one or more of the third to fifth harmonics of the fundamental frequency. A image of the target volume is generated using the detected harmonic components of the received signal.

The present invention relates to imaging systems that use acoustic energy to reveal detail of the internal structure of a subject, such as a human or animal body. In particular, the invention relates to ultrasonic imaging systems for medical applications, suitable for direct imaging of tissue and fluids in the body.

Ultrasonic imaging systems are in widespread use in the medical field for providing images of the internal tissue structure of a patient's body. An excitation beam of ultrasound energy is transmitted into the body tissue and the reflected ultrasound energy echoes are detected, using an appropriate transducer.

During propagation of ultrasound in body tissue, the ultrasound waves undergo gradual distortion in almost every medical application. The distortion is due to slight non-linearities in the sound propagation through the tissue and other body structures, that gradually deform the shape of the wave, and result in the generation of harmonic frequencies which were not present in the transmitted excitation beam. The harmonic frequencies in the reflected beam (echoes) are non-linear and are created when ultrasound waves interact with the tissue.

Until a few years ago, it was widely accepted that these higher frequencies were too small to be measured in the human and animal body. Nevertheless these assumptions became invalid at the frequencies and intensities that became available on biomedical ultrasound equipment. Selective imaging using information contained in second harmonic frequencies proved to be useful in improving ultrasound images. This was found to be particularly true for a class of patients who are technically difficult to image using ultrasound techniques. These technical difficulties may be due to physical characteristics or health conditions such as obesity, pulmonary disease or prior chest surgery. These factors can create haze and other image artefacts and may obscure essential information, such as severe ventricle wall motion abnormalities in stress echocardiography.

This technology, called tissue harmonic imaging, is based on the second harmonic energy at for example 3.4 MHz from an excitation beam frequency at 1.7 MHz. It exploits the gradual generation of the second harmonic frequency as ultrasound waves propagate through tissue. A considerable improvement in image quality is seen.

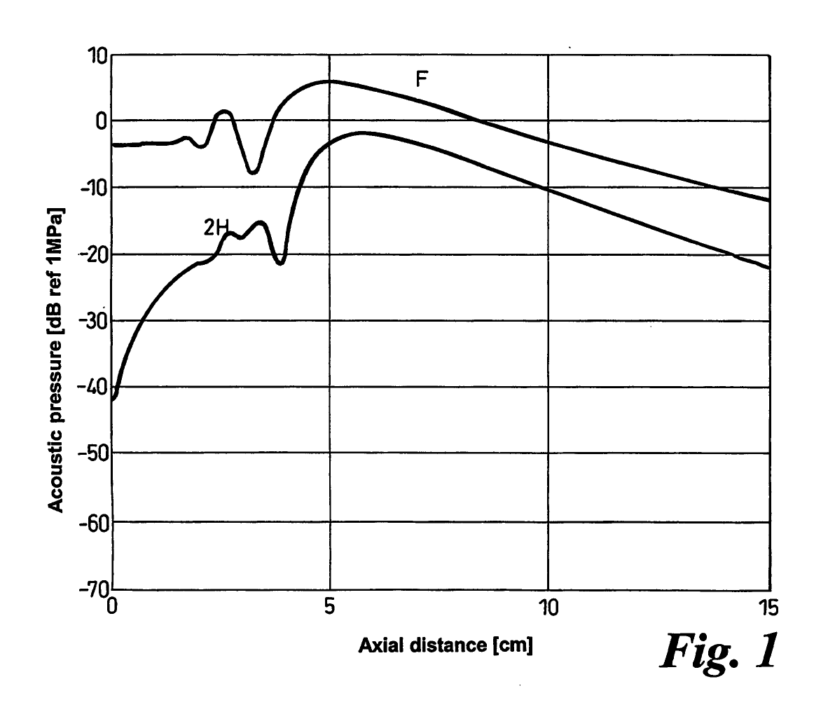

This improvement can be attributed to different mechanisms. Firstly, the amount of second harmonic energy present at the transducer face is small compared to the fundamental energy. The second harmonic component develops gradually as the wave propagates through tissue, and therefore in the nearfield there is only a small amount of second harmonic energy available for reflection from tissue. This is particularly shown in the graph of FIG. 1, which illustrates the ultrasonic acoustic pressure levels of both the fundamental (H) and second harmonic (2H) components as a function of axial distance from the transducer, in the tissue being imaged. Consequently, selective imaging of the second harmonic energy shows less nearfield artefact.

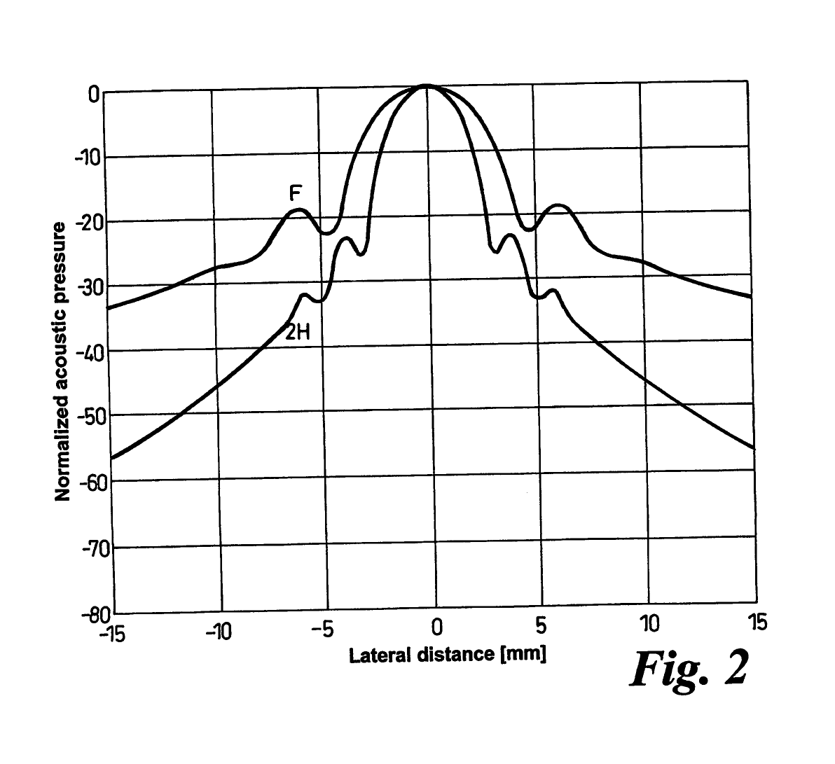

The second mechanism is related to the strength of the second harmonic component. The second harmonic energy is approximately proportional to the square of the energy in the fundamental wave. Thus, most of the second harmonic energy will be concentrated around the strongest part of the fundamental beam (the main lobe), while the weaker parts of the fundamental beam (the grating lobes and side lobes) will generate much less second harmonic energy. This means that the second harmonic beam is less sensitive to clutter and off-axis scatter, giving better and cleaner images. FIG. 2 illustrates the normalised acoustic pressure levels in tissue of the fundamental (F) and second harmonic (2H) beams as a function of distance from the transmitted beam axis.

With the selective use of the second harmonic frequency signal, there is a sacrifice of a certain amount of dynamic range. In current equipment specifications, the mechanical index:
MI=(peak negative pressure in MPa)/√(frequency in MHz))
is permitted to be as high as 1.9. It is known that in most extreme situations, the amount of second harmonic energy returning from tissue is much less than that reflected at the fundamental frequency. The second harmonic signal is 10 to 15 dB less than the fundamental signal. Thus, to use the second harmonic components, there must be excellent sensitivity and dynamic range in the ultrasound receiver to display the harmonic energy without an unacceptable level of thermal noise.

Moreover, the second harmonic energy in the nearfield is also not entirely suppressed and thus is vulnerable to reverberations, particularly arising in ultrasound scans through fatty tissue, ribs and inhomogeneous layers. These problems arise particularly in the class of technically difficult patients discussed above, who yield the worst baseline image. Particular problems in this regard are also encountered when trying to image in awkward locations, for example in cardiological applications while trying to image from a location between the ribs.

The present invention is directed towards a method and apparatus for improved acoustic imaging of tissue within a subject.

According to one aspect, the present invention provides a method for ultrasound imaging comprising the steps of:
transmitting ultrasound energy into a target volume at at least a first fundamental frequency;
receiving reflected and/or scattered ultrasound energy from the target volume; and
detecting components of the received signal at multiple harmonics of the fundamental frequency.

According to another aspect, the present invention provides a method for ultrasound imaging comprising the steps of:
transmitting ultrasound energy into a target volume at at least a first fundamental frequency;
receiving reflected and scattered ultrasound energy from the target volume; and
detecting components of the received signal at one or more of the third harmonic, the fourth harmonic, the fifth harmonic or a higher harmonic of the fundamental frequency.

According to another aspect, the present invention provides an apparatus for ultrasound imaging comprising:
a transmitter for transmitting acoustic energy into a target volume at at least a first frequency;
a receive transducer for receiving reflected and/or scattered acoustic energy from the target volume over a plurality of frequencies; and
a filter for detecting components of the received signal at multiple harmonics of the fundamental frequency.

According to another aspect, the present invention provides an apparatus for ultrasound imaging comprising:
a transmitter for transmitting ultrasound energy into a target volume at at least a first fundamental frequency;
a receive transducer for receiving reflected and/or scattered ultrasound energy from the target volume; and
a filter for detecting components of the received signal at one or more of the third harmonic, the fourth harmonic, the fifth harmonic or a higher harmonic of the fundamental frequency.

Embodiments of the present invention will now be described by way of example and with reference to the accompanying drawings in which:

FIG. 1 is a graph showing the relative acoustic pressure levels in tissue at the fundamental and second harmonic frequencies as a function of axial distance from an acoustic transmitter;

FIG. 2 is a graph showing the normalised acoustic pressure levels in tissue at the fundamental and second harmonic frequencies as a function of lateral distance from the central axis an acoustic transducer transmitted beam;

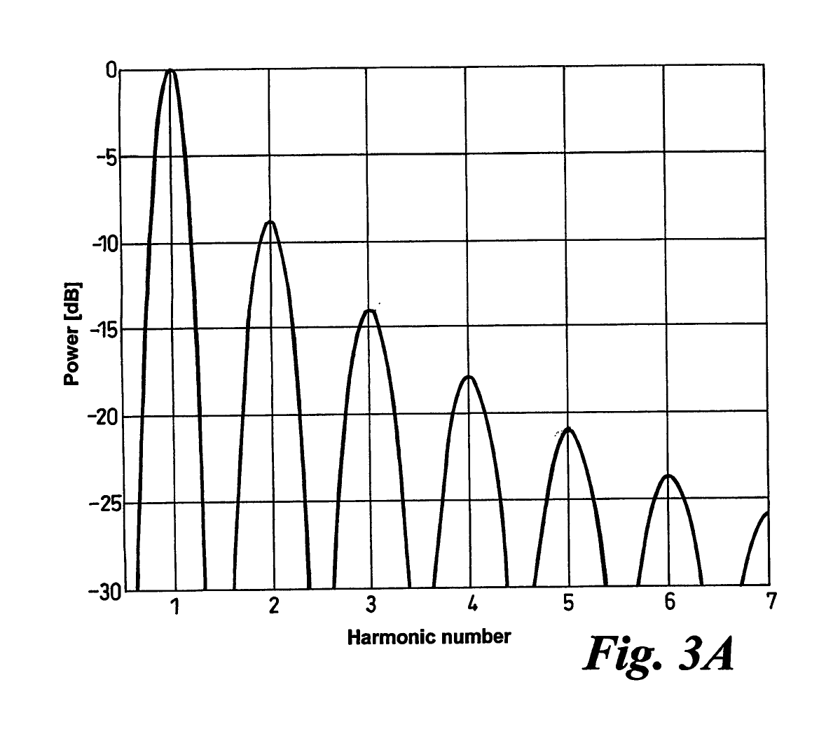

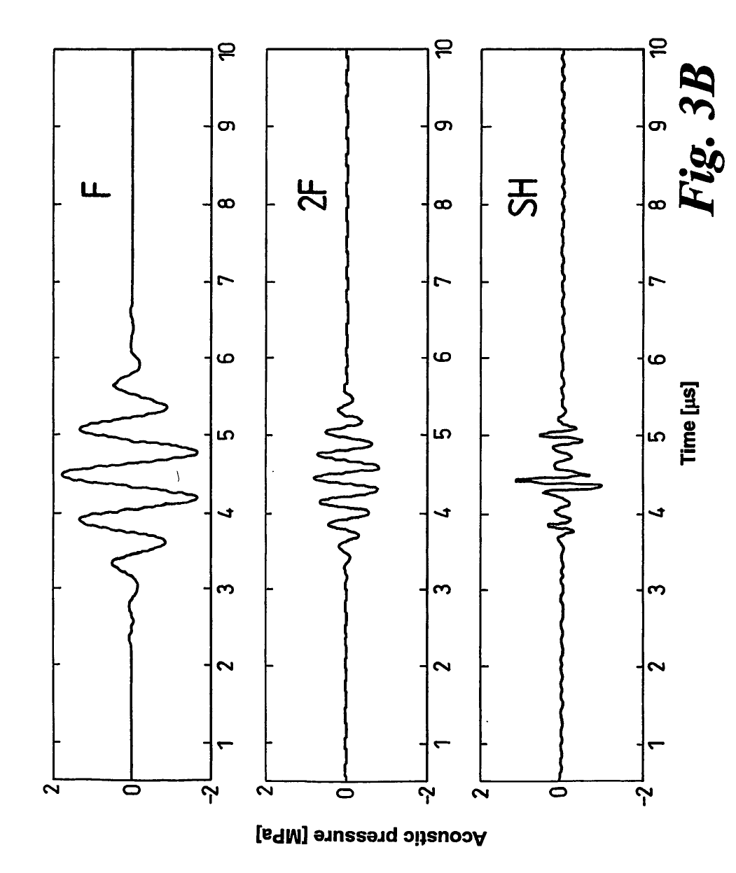

FIG. 3A is a graph showing the relative power levels in a received ultrasound signal as a function of the transmitter frequency (harmonic number);

FIG. 3B is a series of graphs showing the relative acoustic pressure levels in the received ultrasound signal over time as a function of the transmitter frequency;

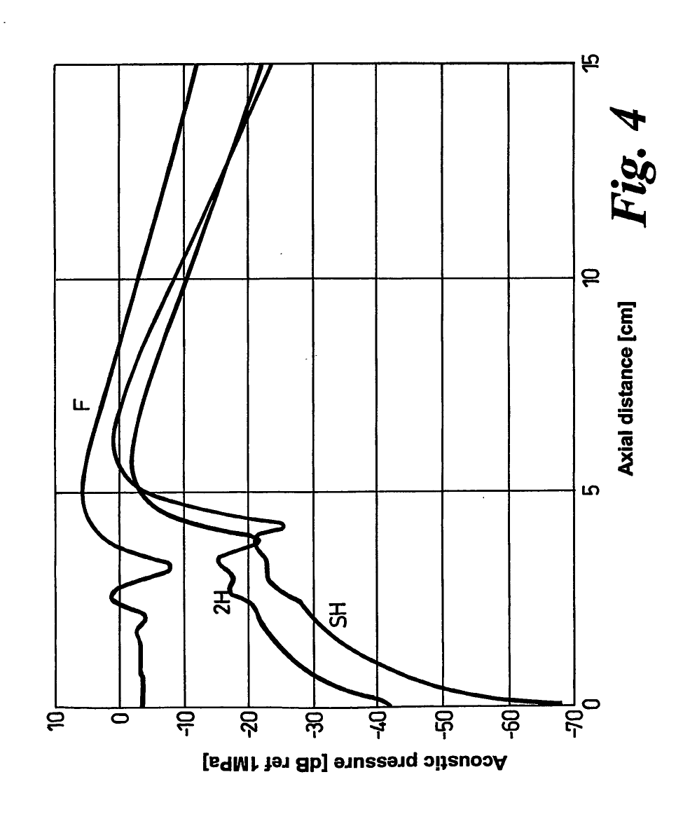

FIG. 4 is a graph showing the relative acoustic pressure levels in tissue at the fundamental (F) and second harmonic (2H) frequencies as a function of axial distance from an acoustic transducer, together with the corresponding level for the combined third to fifth harmonic frequencies (SH);

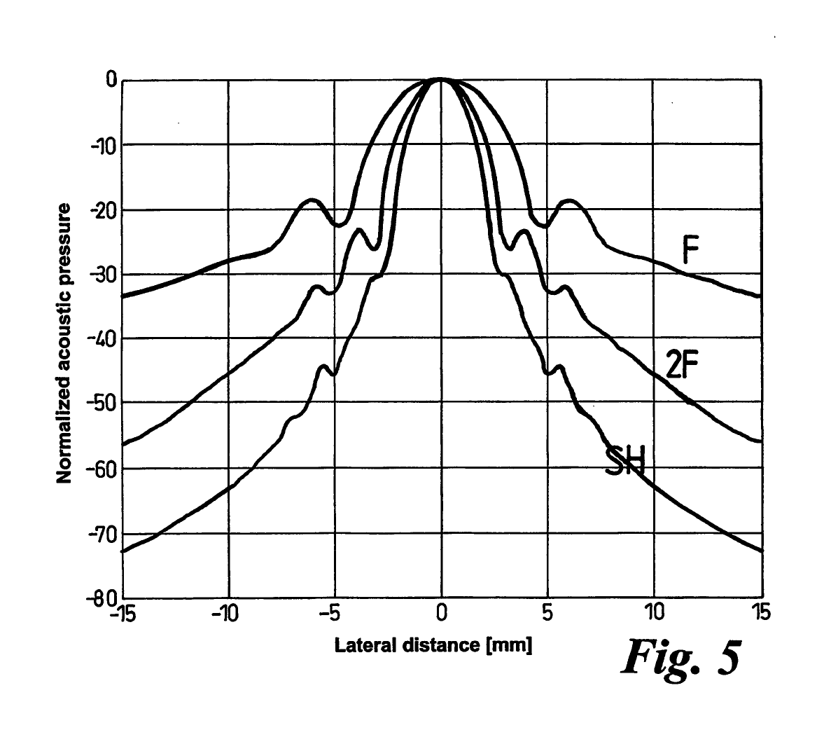

FIG. 5 is a graph showing the normalised acoustic pressure levels in tissue at the fundamental and second harmonic frequencies as a function of lateral distance from the central axis an acoustic transducer beam, together with the corresponding level for the higher harmonic frequencies;

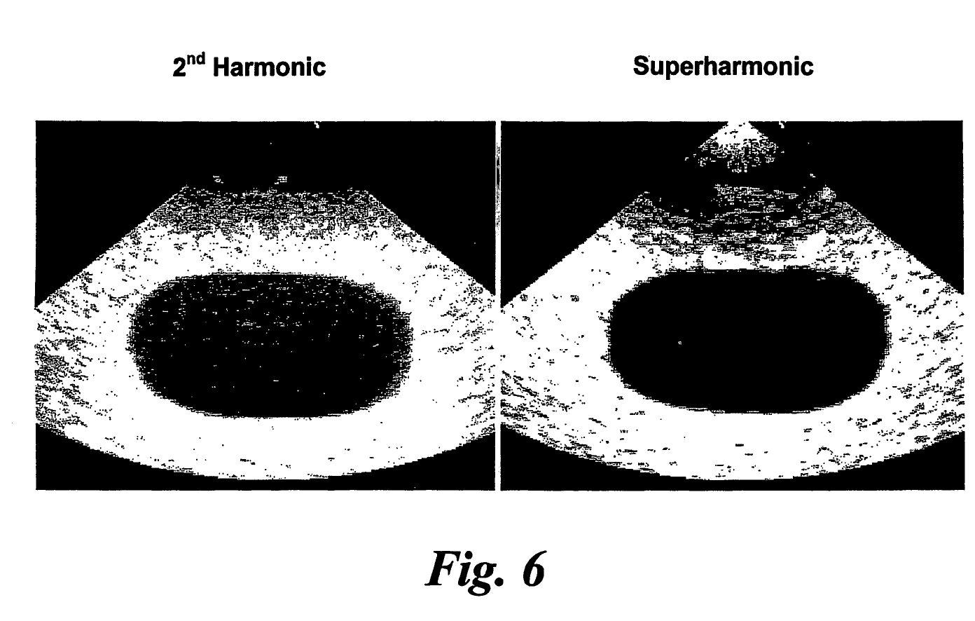

FIG. 6 shows B-mode images of a tissue phantom containing a water cavity using (a) the second harmonic frequency information and (b) the higher harmonic frequencies 3H, 4H and 5H;

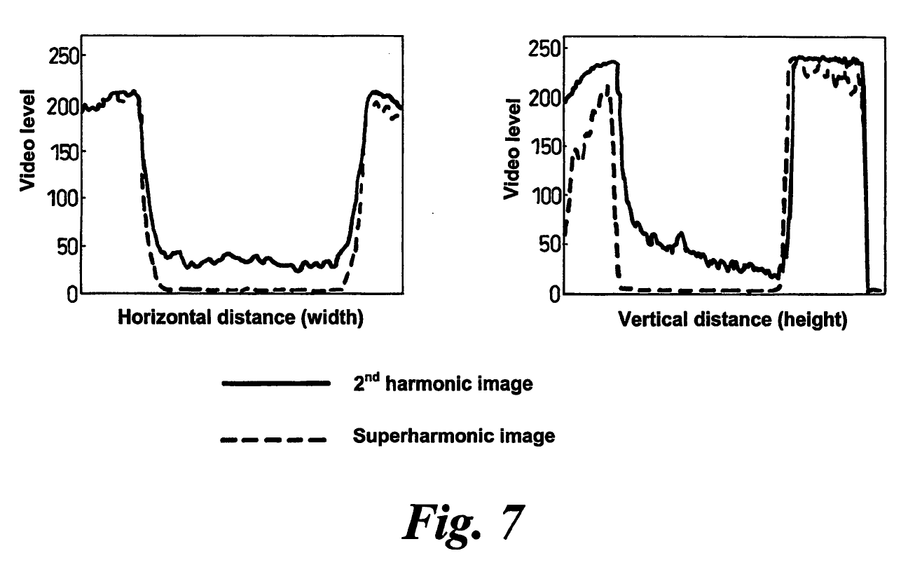

FIG. 7 shows video intensity levels obtained from the images of FIG. 6; and

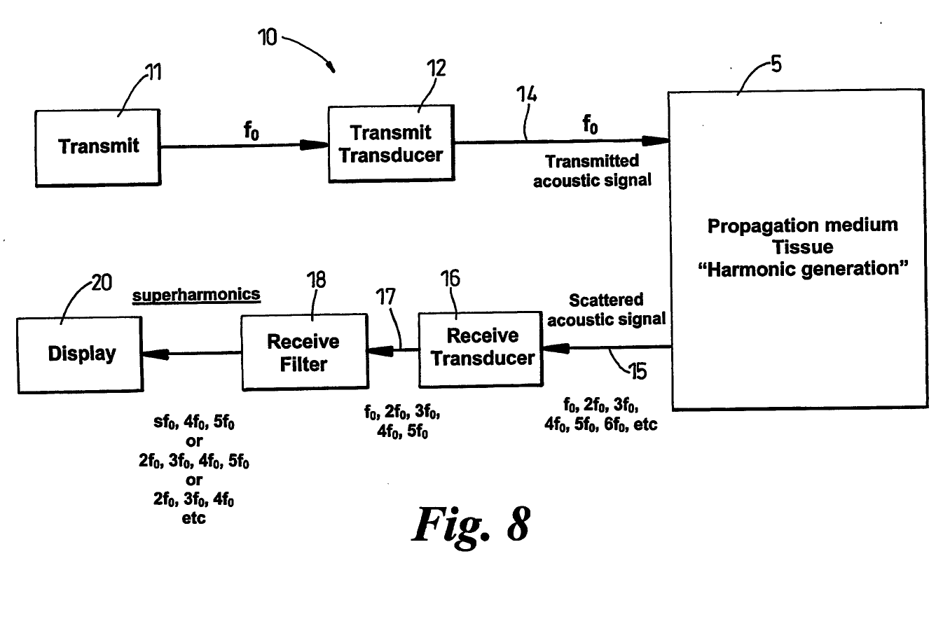

FIG. 8 shows a schematic block diagram of an ultrasound imaging system according to one embodiment of the invention.

According to the present invention, it has surprisingly been discovered that sufficient reflected and scattered ultrasound energy can be received directly from tissue and fluids in a target body to be useful in imaging applications, ie. without the use of contrast agents. Therefore, the present invention is particularly applicable for direct ultrasound imaging of tissue and fluids within a body without the deliberate introduction of contrast enhancing agents such as gas bubbles or particulate material into the body being imaged which otherwise contribute significantly to reflected and scattered ultrasound energy.

With reference to FIGS. 3A and 3B, it will be observed that at available excitation pressures the levels of harmonic components generated in the tissue and reflected to the transducer are relatively high. It is clear that a significant amount of energy is converted and transferred from the fundamental frequency to the harmonic frequencies up to at least the fourth and the fifth harmonic components. The examples shown are obtained using a transmit (excitation) frequency of 1.7 MHz, the medium being tissue with the following physical characteristics: attenuation coefficient alpha=0.7 dB/cm/MHz and non-linearity coefficient (B/A)=8.5. The mechanical index (MI) used was 1.3.

In the received signal, the second harmonic component is approximately 8 dB below the fundamental component and the fifth harmonic is only about 20 dB below the fundamental component. The higher harmonic components represent additional information, which can be more relevant and with characteristics that are superior to the second harmonic alone.

A presently preferred technique to take advantage of the higher harmonics and to bring all the information together is to combine and incorporate all the higher harmonics into a single component which we will refer to as the superharmonic component.

The superharmonic component can have different combinations. It may include the third harmonic, the fourth harmonic and the fifth harmonic all together, which can be combined at the detector using a wide band frequency filter. Alternatively, the superharmonic component may comprise the second harmonic frequency up to the fifth harmonic frequency. Other harmonic combinations, such as 2+3+4, 3+4, 2+3, etc are within the scope of the present invention.

The extra information that is brought by the superharmonic component when compared to the second harmonic alone provides different advantages for ultrasound imaging.

First, the distortion of the ultrasound wave is gradual, which means that the harmonic frequency energy rises with propagation distance. As shown in FIG. 4, at the transducer surface, the second harmonic energy is about 35 dB below the fundamental energy whereas the superharmonic energy is about 70 dB below the fundamental energy.

The relatively low level of the superharmonic energy upon entry to the body being imaged means that imaging artefacts caused by reverberations at boundaries in the body being imaged are significantly reduced or eliminated. For example, when imaging through a narrow acoustic window, such as between the ribs, the artefacts introduced by the ribs (clutter, noise, reverberations, haze) may be substantially reduced or eliminated entirely using the superharmonic component.

The second advantage of using the superharmonic component is that it builds up with transmission distance. Even though the superharmonic component (3H+4H+5H) is much lower than the second harmonic component at the chest wall, it builds up so fast (see FIG. 4) that at imaging distances of a few centimetres, enough superharmonic energy has been generated from the fundamental beam to yield a significant superharmonic component. Also as shown in FIG. 4, at imaging distances of 5 cm in the example given, the superharmonic component surprisingly is even higher than the second harmonic component.

A third advantage of the use of the superharmonic component is the substantial removal of off-axis echoes. As shown in FIG. 5, the generation of superharmonic components is substantially confined to the strongest part of the fundamental beam, even more so than is the second harmonic. This has the beneficial effect that the superharmonic beam width is much narrower than the second harmonic beam width. The beam width at the superharmonic frequency is found to be half of the transmitted fundamental beam width, whereas the second harmonic beam width is only 30% narrower. For example, for a beam width of 5.3 mm (around the focal point), and 3.5 mm at the second harmonic, the superharmonic components (3H+4H+5H) have a beam width of less than 2.6 mm. The narrow beam width using the superharmonic component results in a substantial enhancement in lateral resolution, eg. by a factor of two. Moreover, the superharmonic energy is substantially concentrated to the main beam and shows much lower side lobe and grating lobe levels.

A fourth advantage of the use of the superharmonic component is that problems associated with the spectral overlap between the transmitted and received signals is substantially eliminated. In second harmonic imaging systems, the spectral overlap between the fundamental frequency band and the second harmonic frequency band has to be reduced. This impairs the imaging resolution. Consequently a compromise is mandatory between the resolution and the sensitivity of the system. This compromise is not required in the superharmonic system since the receive frequency band is remote from the transmit frequency band. Moreover, since the receive frequency band is wide (eg. covering the third to fifth harmonics or more), the axial resolution is further increased.

Typically, in commercially available machines, the transmit frequency is a band of frequencies resulting from transmitting an excitation signal at the fundamental frequency containing 2.5 or 3 cycles. This means about 60% to 70% bandwidth either side of the centre frequency.

The superharmonic component generation is a highly non-linear process implying that only fundamental energy (and some second harmonic energy) above a certain level will give rise to superharmonic energy. This is critically important in many applications.

Selective superharmonic imaging will yield a dramatically cleaner and sharper contrast between different structures in the subject being imaged. FIG. 6 shows two B-mode images of a tissue phantom containing a water cavity. The left-hand image was made using standard second harmonic imaging mode using a standard, commercially available probe. The transmit frequency was 1.7 MHz, and the MI=1.3. The right-hand image was made using a superharmonic mode, combining the third to fifth harmonics (3H+4H+5H). The transmit frequency was 1.2 MHz, and the MI=1.0. The water cavity appears much darker and more sharply defined in the superharmonic image than in the second harmonic frequency image.

The combining of the higher harmonic components into a single superharmonic component is beneficial way of increasing the amount of detected energy compared to the second harmonic energy alone. Indeed, the distortion of the ultrasound waves in tissue is the mechanism by which energy is transferred to the harmonic frequencies and this energy is decreasingly split between all the harmonics. Use of the superharmonic energies effectively allows recovery of otherwise lost energy due to distortion into a usable and valuable information source.

The superharmonic energy level is often higher than the second harmonic energy level and in some situations can be even higher than the energy level in the fundamental component. For tissue harmonic imaging, the super-harmonic beam offers better sensitivity, higher resolution and improved signal to noise ratio.

The threshold for superharmonic generation depends on frequency and acoustic pressure (MI). In practice, an MI value above 0.1 or 0.2 is required in order to generate superharmonic components that have sufficient energy to be detectable.

FIG. 7 shows the video intensity level obtained from the images shown in FIG. 6. The left-hand graph shows the video level across a horizontal cut (made at the middle of the image in the horizontal plane), comparing the second harmonic image (solid line) and the superharmonic image (dashed line). The right-hand graph shows the video level across a vertical cut down the centreline of the image, again comparing the second harmonic image (solid line) and the superharmonic image (dashed line).

FIG. 7 illustrates the high contrast of edges of structures (the water cavity) detected and displayed using the superharmonic frequencies compared to the second harmonic frequencies. Moreover, the video level inside the cavity is much darker (black video level=0) in the superharmonic image than in the second harmonic image. The difference is up to 25%. These improvements result in much better edge detection (eg. in cardiology the detection of the left ventricle contour is of considerable importance), since the cavity is much darker and the walls are more sharply displayed. More generally, images generated using superharmonics have exceptionally improved clarity without the use of contrast agents.

With reference to FIG. 8, to utilise the methodology described above, a preferred embodiment of the present invention provides a wide band ultrasound imaging system 10. A transmit signal generator 11 is used to generate a transmit signal at frequency f₀ which is supplied to a transmit transducer 12 in conventional manner. Transducer 12 insonifies the object 5 under analysis, by transmission of an ultrasound excitation beam 14 at frequency f₀ into the object 5. The object is typically the human or animal body.

Within the object 5, propagation of the transmitted beam 14 results in generation of harmonics in the scattered and reflected beam, which therefore comprises energy at the frequencies f₀, 2f₀, 3f₀, 4f₀, 5f₀, 6f₀ etc.

A wide band receive transducer 16 detects the reflected acoustic signal and converts the acoustic energy into an electrical signal 17, including at least the fundamental frequency f₀ up to the fifth harmonic 5f₀. This is filtered according to a desired strategy, to extract signals in one or more frequency bands, eg. 3f₀+4f₀+5f₀; or 2f₀+3f₀+4f₀+5f₀; or 2f₀+3f₀+4f₀, etc. A preferred transducer is a dual frequency probe.

Such a dual frequency probe was used to generate the images of FIG. 6. Preferably, the transducer transmits an excitation beam in the range 1 MHz to 10 MHz. In another preferred embodiment, the transducer transmits at 1.2 MHz and receives in the frequency band 3.6 MHz to 6 MHz. In another preferred embodiment, the transmit transducer and the receive transducer are provided in a single broadband transducer probe. Echo signal samples may be delayed by a beam former to form a coherent signal.

The receive filter 18 may be of any type suitable for the purpose, such as an FIR filter comprising a series of multipliers and accumulators and a controller to weight the multiplying factors. Other signal processing techniques besides filtering may be used to separate the superharmonic components from the received echo information.

A suitable signal processor and display device 20 is used to generate an image using the received superharmonic signals. In a preferred embodiment, the signal processor 20 uses the selected harmonics only.

In another embodiment, the image may be generated using information from one or more harmonics, subsequently processed or refined using information from other harmonics. For example, image information may be gathered from two superharmonic combinations, such as (2H+3H), and (4H+5H), and a compound (composite) image generated using the average, sum or difference.

The superharmonic imaging system 10 may incorporate a Doppler processor for Doppler processing according to conventional techniques.

In summary, the use of superharmonic components as described herein provides at least the follow advantages. Superharmonics have almost no energy in the nearfield, resulting in minimal or no reverberations or multipath reflections. Sidelobes and grating lobes of the superharmonic components are much lower than fundamental and second harmonic components, resulting in much reduced clutter and noise from off-axis scatterers. The superharmonic energy at typical imaging distances in the human body is higher than the second harmonic energy, and in some situations (eg. SH=2H+3H+4H+5H), can be even higher than the fundamental energy. This results in a much higher signal to noise ratio and sensitivity. For superharmonic imaging, axial and lateral resolutions are better than for second harmonic imaging and the compromise between sensitivity and resolution does not exist.

1. A method for ultrasound imaging comprising the steps of:
transmitting ultrasound energy into a target volume at at least a first fundamental frequency;
receiving a signal of reflected and/or scattered ultrasound energy from the target volume; and
detecting components of the received signal at multiple harmonics of the fundamental frequency.

2. The method of claim 1 further comprising generating an image of the target volume using the multiple harmonic components of the received signal.

3. The method of claim 2 wherein the detected components comprise the second harmonic and at least one higher harmonic.

4. The method of claim 2 wherein the detected components comprise the third harmonic and at least one higher harmonic.

5. The method of claim 4 wherein the detected components comprise the third, fourth and fifth harmonics.

6. The method of claim 8 applied in the direct imaging of at least one of tissue and fluids in a body.

7. The method of claim 6 applied in the direct imaging of tissue in a body.

8. The method of claim 6 applied without the use of artificial contrast agents introduced into the body.

9. A method for ultrasound imaging comprising the steps of:
transmitting ultrasound energy into a target volume at at least a first fundamental frequency;
receiving a signal of reflected and scattered ultrasound energy from the target volume; and
detecting components of the received signal at one or more of the third harmonic, the fourth harmonic, the fifth harmonic or any higher harmonics of the fundamental frequency.

10. The method of claim 9 further comprising generating an image of the target volume using only the detected harmonic component.

11. The method of claim 10 wherein the detected components relate to received ultrasound energy form tissue.

12. (canceled)

13. (canceled)

14. (canceled)

15. (canceled)

16. (canceled)

17. (canceled)

18. Apparatus for ultrasound imaging comprising:
a transmitter for transmitting acoustic energy into a target volume at at least a first frequency;
a receive transducer for receiving reflected and/or scattered acoustic energy from the target volume over plurality of frequencies; and
a filter for detecting components of the received signal at multiple harmonics of the fundamental frequency.

19. The apparatus of claim 18 further comprising signal processing means for generating an image of the target volume using the multiple harmonic components of the received signal.

20. The apparatus of claim 18 wherein the filter is adapted to detect the second harmonic and at least one higher harmonic of the fundamental frequency in the received signal.

21. The apparatus of claim 18 wherein the filter is adapted to detect the third harmonic and at least one higher harmonic of the fundamental frequency in the received signal.

22. The apparatus of claim 21 wherein the detected components comprise the third, fourth and fifth harmonics.

23. (canceled)

24. (canceled)

25. (canceled)

26. (canceled)

27. (canceled)

28. (canceled)

29. (canceled)

30. The method of claim 1 wherein the detected components comprise the second harmonic and at least one higher harmonic.

31. The method of claim 1 wherein the detected components comprise the third harmonic and at least one higher harmonic.

32. The method of claim 31 wherein the detected components comprise the third, fourth and fifth harmonics.

33. The method of claim 6 applied without the use of artificial contrast agents introduced into the body.
